(19) United States
(12) Patent Application Publication
(10) Pub. No.: US 2021/0127981 A1
(43) Pub. Date: May 6, 2021

(54) DETERMINATION AND CORRELATION OF FLOW STATES

(71) Applicant: Suzanne Brown, Zurich (CH)

(72) Inventor: Suzanne Brown, Zurich (CH)

(21) Appl. No.: 17/150,955

(22) Filed: Jan. 15, 2021

Related U.S. Application Data
(63) Continuation-in-part of application No. 17/008,181, filed on Aug. 31, 2020.
Application No. 17/008,181 is a continuation-in-part of application No. 16/730,183, filed on Dec. 30, 2019.
(60) Provisional application No. 62/894,472, filed on Aug. 30, 2019.
(60) Provisional application No. 62/787,078, filed on Dec. 31, 2018.

(51) Int. Cl. A61B 5/02; A61B 5/00

(52) U.S. Cl. CPC A61B 5/02; A61B 5/7246; A61B 5/6801

(57) ABSTRACT

A method includes receiving, from a sensor, a signal associated with a user. The method further includes determining, by a processing device, based on the signal, that the user is in a flow state.

### REFERENCE TO RELATED APPLICATIONS

This application claims the benefit of U.S. patent application Ser. No. 17/008,181, filed Aug. 31, 2020, which claims the benefit of U.S. Provisional Application No. 62/894,472, filed Aug. 30, 2019, and further claims the benefit of U.S. patent application Ser. No. 16/730,183 filed Dec. 30, 2019, which claims the benefit of U.S. Provisional Application No. 62/787,078, filed Dec. 31, 2018, all of which all hereby incorporated by reference in their entirety.

### FIELD OF TECHNOLOGY

The embodiments described herein pertain in general to determination and correlation of flow states and corresponding software and hardware platforms.

### BACKGROUND

When a person is experiencing Flow (e.g., a flow state), the cells in her body, brainwaves in her mind and magnetic fields radiating from the cells and organs in her body are more coherent or have a resonate frequency and/or carry a stronger amplitude.

### BRIEF DESCRIPTION OF THE DRAWINGS

The described embodiments and the advantages thereof may best be understood by reference to the following description taken in conjunction with the accompanying drawings. These drawings in no way limit any changes in form and detail that may be made to the described embodiments by one skilled in the art without departing from the spirit and scope of the described embodiments.

FIG. 1 is a block diagram illustrating a variety of wearables into which a flow sensor may be incorporated, in accordance with some embodiments.

FIG. 2 is a first block diagram illustrating a variety of devices into which a flow sensor may be incorporated, in accordance with some embodiments.

FIG. 3 is a second block diagram illustrating a variety of devices into which a flow sensor may be incorporated, in accordance with some embodiments.

FIG. 4 is a third block diagram illustrating a variety of automotive-related devices into which a flow sensor may be incorporated, in accordance with some embodiments.

FIG. 5 is a fourth block diagram illustrating a variety of devices into which a flow sensor may be incorporated, in accordance with some embodiments.

FIG. 6 is a block diagram of an example computing device 600 that may perform one or more of the operations described herein, in accordance with some embodiments.

FIG. 7 is a flow diagram of method that may perform one or more of the operations described herein, in accordance with some embodiments.

### DETAILED DESCRIPTION

There is tremendous value in anyone who achieves a coherent resonate flow state mentally, emotionally, physically and/or spiritually or even a simple lack of resistance (“Flow” or “Flow State”). In one embodiment, Flow may be correlated with a state of love and appreciation. This may be the basis of the value of the new token described herein, and may be based on each person's ability to focus on feeling love, appreciation, and blessed.

When a person is experiencing Flow, the cells in her body, brainwaves in her mind and magnetic fields radiating from the cells and organs in her body are more coherent or have a resonate frequency and/or carry a stronger amplitude. A Flow State can be measured by any number of methods described herein to measure resonance or alignment with one's inner or higher true self or soul, which may be indicated by goose-bumps, tingles, higher frequencies, more radiance and lower resistance. A person in Flow will have the intangible quality of feeling good emotionally, mentally or physically. Their intuition and clarity is heightened. They are far less prone to making errors. In fact, they are more likely to perform better mentally and physically at peak levels. They feel more peaceful and inspired in their lives. They are happier and more fulfilled in life with greater clarity and satisfaction. They feel self-empowered and have greater capacity for love, compassion and appreciation. They are physically healthier and more vibrant and alive. Someday, we may discover that a person in Flow is literally flowing more source energy (invisible life force) that literally gives and sustains life. A person who consistently achieves higher Flow States has significant value and greater leverage to influence their life for the better and those around them in a positive, rewarding and uplifting way. These embodiments include methods for measuring and validating when a person is in a Flow State and then associate Flow States with anything that may have contributed to or coincide with reaching a Flow State and then issue a reward to the other parties involved. The result is measuring the value of physical goods, services and any content by their ability to contribute to a person reaching Flow States of greater satisfaction and performance. These embodiments are further qualified by the type of Flow achieved and provide feedback mechanisms to the parties involved and the associated products and services. Mutual interests between providers and consumers will be better aligned and their relationship closer, deeper, more authentic, more valuable and farther reaching. Product and content sales will have an ongoing revenue stream and no longer be limited to one-time payments or short-lived returns. The economics of things, services and experiences will never be the same. Good intentions will pay off and more valuable goods and services will be created and rewarded in an upward spiral. Win-Win will prevail. Everything else will become obsolete. When the tide rises, all the boats float.

The embodiments provided herein are related to the determination and correlation of flow states, and more particularly, are related to systems and methods for determining flow states and associating/correlating anything with flow and issuing rewards and/or feedback, and the possibility of sharing the value of your flow state with others. The embodiments are further defined to determine the authenticity and therefore the inherent value of any content or creation.

The intention of the embodiments described herein is to enhance human life experience on earth in the most beneficial and positively impactful way. We will do this by focusing on embodiments described herein that may increase the likelihood of a person to reach, experience, and extend Flow States with anything that can be correlated, give feedback, and provide mechanisms to reward all things correlated, including “non-things” like music, videos, words, content, art, kindness, compassion, love, intentions, etc. These embodiments may associate people in Flow States with their surroundings, actions, content, or anything in space and time that may be related and thereby correlated to such Flow States. These embodiments will enhance the awareness, relationship, and appreciation between themselves and what may have contributed to Flow States. Furthermore, these embodiments may show how such Flow States positively benefit the achiever and influence all things and people around them or connected to them (even remotely) in a beneficial way. These embodiments may increase a person's ability to reach, increase, and sustain Flow States with some feedback, incentives, or rewards to the person and anything that can be associated with a person in a Flow State. The rewards may be embodied in a virtual representation of value (e.g. digital token) or any form of monetary value, revenue-share, or payment in traditional fiat currencies or crypto-currencies. The fully realized intention of these embodiments may fundamentally and positively change the economics of physical goods and non-physical content to share appreciation and value with those who benefit and inspire value in others and thereby create win-win incentives between people, products, content and companies.

A Flow State may be one of the most desired states anyone can experience and therefore embodies tremendous value for the experiencer and anyone influenced by them or connected to them (even from a distance) through uplifting intentions. In one embodiment, a person in a Flow State may experience extra-ordinary clarity, intuition, insights, inspiration, and may receive great ideas, inventions, and win-win solutions. Their body and all the cells in their body will begin to work together in greater coordination and coherency. Sickness will fade, healthiness and vitality prevail. Mental and physical performance will excel. A person experiencing Flow will perform at their best, is more productive, more effective, and has more positive influence on other people. A person in a Flow State will have better timing, often seemingly miraculous to those watching. A person experiencing Flow will feel happier, more fulfilled, worthier, peaceful, compassionate, appreciative, and loving. Their physically body will function better, stronger, and healthier. The benefits of Flow may be intangible and intrinsic at first. They may be mental or emotional internally, but highly valuable intrinsically which eventually manifests externally over time in desirable ways, often for everyone, but in particular for the person in Flow. Where focus goes, energy Flows. What is the energy comprised of that which is flowing and how can we measure it? Turn it on? Make it last? Share it with others? The answers to these questions are the essence of these embodiments.

Example embodiments achieving Flow States may be random, mysterious and short-lived with the majority of people today. Most do not know how to deliberately calibrate, achieve or retain a Flow State to enjoy or realize the benefits to a significant degree. The average person accidently experiences Flow while driving their car in a semi-meditative state. A more self-aware person may reach a Flow State while meditating on purpose to quiet their mind and may later enjoy a more receptive and lasting Flow State. Inventors, song-writers, and other creative endeavors are more prone to making discoveries and writing new hit songs while in or shortly after experiencing a Flow State. Many athletes get there by calibrating and focusing their physical and mental alignment in the most coordinated and perfect way resulting in winning shots, fastest time, and gold medals. Almost everyone has experienced a short lasting Flow State during a sexual orgasm. Some have more lasting Flow States while focusing on someone or something they deeply appreciate or love. Some experience Flow while listening to music or experiencing something that inspires them and gives them goosebumps. The embodiments described herein raise the bar for measuring, achieving, retaining and rewarding Flow States in order to realize longer-lasting benefits and to derive tangible and intangible benefits and results. The embodiments derive greater value by enhancing and sharing the value of Flow with others. Flow States with these embodiments may become more commonplace, more retainable, returnable, and more valuable with more tangible and sharable results. More people will begin to come to understand how to achieve Flow States and how to make them last longer and reach higher stable levels. More people may realize how to deliberately achieve Flow States. With these embodiments, more people may be able to realize and materialize the value of Flow States in a more tangible and satisfying ways by using virtual and physical tokens that embody the very essence of their true value. More people will begin to think differently about how they are creating and adding value to their lives and those around them in a more meaningful and satisfying way and the world at large. More people may feel more worthy, more inspired, more valuable, more lovable, more peaceful. More people may achieve self-actualization, the top of Maslow's hierarchy of needs and be more likely to do what they are best at, what they were born to do, and realize their dreams. The world and people will begin to transform from the inside out. They may realize their purpose and reason for being. They may feel more authentic, happier, more fulfilled and connected in a more satisfying way. Peace, love and joy may become the new normal on earth. This will become the state of the art of becoming superhuman.

Some of the embodiments herein stand on their own while many are a natural extension to those filed on 31 Dec. 2018 entitled “VIRTUAL REPRESENTATION OF INTRINSIC VALUE” under U.S. Application Number 2787078 by the same author and inventor. When a person has achieved a Flow State, especially a higher one and/or a lasting one, this has tremendous value. The former patent filing describes many ways of achieving and measuring Flow States. The novelty in the embodiments therein embody the value of Flow States by issuing a virtual or physical token and thereby making it more tangible, measurable, transferrable, sharable, financial and impactful in nature.

The embodiments herein this application take these further by making associations or correlations with anything that may have contributed to or be associated with your Flow State. What were you watching or listening to when you reached a Flow State? What were you doing? Where were you? Who were you with? What shoes were you wearing when you broke the world record? What skis were you skiing on when you had your best time? What tennis racquet were you using when you hit those winning shots during match point in a Flow State and won the Grand Slam title? What guided meditation or teacher were you listening to when you attained that higher lasting Flow State? What music were you listening to? What concert were you attending? What book or article were you reading? What movie or show were you watching? What were you thinking? What were you reading? What were you writing? Who or what were you appreciating or loving? Knowing the answers to these questions and providing a mechanism to automatically associate, measure and reward them for their correlation has tremendous value to all parties involved.

All interests become more aligned in a win-win system. The person achieving Flow State has more valuable feedback on what works best for them. The contributor can also receive feedback and be rewarded for their contribution for inspiring Flow in others. All parties become more focused on what adds value and who and what they value and a way to amplify it and share it. Incentives become mutually beneficial and valuable. Makers of physical goods and/or providers of content and services will be able to focus better on improving their goods and services to improve their customer's user experience, performance and satisfaction. The relationship between providers and consumers becomes closer, longer lasting and more meaningful and more valuable as it literally pays off. One-time transactions may become a thing of the past. All makers of physical goods will have new methods and incentives for receiving on-going residual returns whenever their customers achieve Flow States with their products.

Product makers will become more far-sighted and less short-sighted with their products and serving customers. All creators of content and services will have new methods and incentives for receiving on-going residual returns whenever their consumers achieve Flow States with their content or services, even when such content is distributed in unauthorized ways. This may naturally attract makers, creators and consumers to more authentic and valuable products and services and detract from fraudulent copies that don't pay off and won't have residual value for either party. People and stuff get better . . . naturally. Lesser quality products and content become obsolete . . . naturally. The economics change for the better for all parties involved. The back-end becomes the front-end. The intrinsic value of a product or service becomes more transparent and more valuable and paradoxically more tangible. Now makers of physical goods and content can better realize their value and contribution by being issued a reward for their products, services and content in association with someone achieving a Flow State using their products or experiencing their content if it truly adds value to the recipient.

The intrinsic value of achieving a Flow State can now be created and stored in digital or physical tokens. Tokens can now be created and time-stamped during a certain event, e.g. Rafa Nadal creating tokens while in the Flow while playing in a tennis match against Roger Federer. Occasionally, some of these tokens are created at virtually the same time or during the same tennis match resulting in a unique blended Token of both of them, an “Alloy” Token. Alloy tokens become more valuable and commemorative when more are created during the same event. Paradoxically, in a match where more are created, they are each likely to be worth more, not less. This logic is the opposite of money and valuables in the world today of zero-sum where scarcity and lack make something seem worth more. For tokens based on something with intrinsic non-tangible value like Flow which is equivalent to truth, peak performance, love, appreciation, clarity, inspiration), the more, the better. These tokens are win-win, so more is worth more. The value of money today is based on debt and therefore win-lose, therefore creating more money makes them worth less.

Flow States can now be associated with another person, experience, place and thing. While listening or watching content that inspires someone to Flow, the creator of the content also receives a token of themselves or of the person who got into Flow by listening or watching their content, or any combination thereof. For example, while attending a PINK concert, she receives tokens of herself and/or of individuals in the audience who get into Flow while attending, watching, dancing and listening to her, or a member of the audience receives a token of PINK when s/he Flows at the same time or the same concert with PINK, or any combination thereof to share and inspire each other. In another example, while playing tennis, Roger Federer gets into Flow while playing with a Wilson tennis racquet triggering a WILSON Flow token to Wilson or perhaps a ROGER token to Wilson or a general purpose Token of Me (“TOM token”) to both parties or shared between them or any combination thereof. Wilson can now advertise that more athletes get into Flow while playing with Wilson equipment than any other racquet. High performance can now be measured and validated in a new way. A little known fact is that the average person accidentally gets into a Flow state while driving their car because they are gently focused and using little effort. Driving can induce a meditative Flow State. So, while driving say a Tesla, the driver gets into a Flow State, which triggers a TESLA token and/or a token of the driver herself, or a TOM token to Tesla or any other combination thereof. Tesla can now advertise that more drivers get into Flow while driving a Tesla than any other vehicle. Tesla can prove the value by showing how infrequently a driver has an accident while in Flow. Insurance companies can reward safer Flow drivers. Sensors are being placed in automobiles to measure and monitor our driving to offer discounts on our insurance for safe drivers, but what constitutes a “safe” driver? The problem with today's technology is their assumptions on data looking to measure smooth driving within speed limits. So, the little old lady who drives too slow and doesn't look before moving into your lane will be rewarded while the confident race car driver who swerves out of her way or slams on the brakes to avoid hitting grandma may be charged more for his erratic driving behavior. The intention on these embodiments are to measure and correlate a more accurate and true assessment of safe driving based on Flow states or those close to or related to Flow.

These new embodiments may be based on a “Flow Sensor” built into any device worn or carried by the user (e.g. necklace, scarf, headband, hat/helmet/visor, glasses, necklace, earrings, piercings, wristband, bracelet, watch, ring, belt, belt-buckle, buttons, clothing, shoes, cell phone, key fob/accessory, etc.) and/or built directly into sports equipment, steering wheels or seats in vehicles, motorcycles, bicycles or scooters, computers, even a patch worn on the skin or even implanted inside the body. Such Flow Sensors may be developed by a variety of manufacturers using the embodiments herein or otherwise. Regardless of how the Flow State is measured or determined by any Flow Sensor, any validation or reward issued for the Flow State may be covered by the embodiments described in provisional patent Application Number #2787078 and any coupling of rewards with anything associated with the Flow State may be covered by the embodiments described herein this patent application.

The Flow Sensor embodiments herein may expand on the methods for measuring and/or validating Flow States. The former U.S. provisional patent Application Number 2787078 concentrated on measuring magnetic fields of the body and in particular the heart or the brain using nitrogen-doped diamonds or measuring heart rate variability (HRV). The present embodiments expand further on the methods by which a Flow Sensor may measure Flow States and how they are validated.

Goose Bumps/Tingles/Skin Piloerection

When a person experiences the sensation of emotionally inspired goose bumps, tingles or chills (not from a fever or from being cold suddenly), but rather from an auditory or mental/thought perception that truly agrees with your core and profoundly gives rise to inspiration and resonance within your soul—this is a very powerful indicator of Flow State. When a person listens to music or hears someone say something that resonates as true within you, this can also trigger goose bumps, tingles or chills. The inventor believes this happens when your true self is focused on the truth in resonance with your soul or source within and may be related to feelings of self-actualization and true self-worth. Let's call these Flow-induced emotional goosebumps “Tingles.”

There are many ways to measure Tingles, which can involve the stimulation of the body's psychological and physiological systems. Tingles can be elicited by the stimulation of a number of sensory inputs including aural, tactile, visual, gustatory stimulation and mental thoughts by measuring physiological responses such as heart rate, skin conductance and breathing rate. Empirical studies have indicated that Tingles are coupled with an increase in electrodermal activity (EDA) or Skin Conductance Response (SCR) due to activation of the sympathetic nervous system (SNS). Furthermore, studies have shown that Tingles are associated with enlarged pupils, which strengthens the notion that Tingles are related to the SNS. Therefore, the Flow Sensors may include measuring EDA or SCR and/or coupling this information with heart rate variability, breathing rate, pupil dilation or increase in dopamine neurotransmitters or a number of other measurements. Positron emission tomography (PET) scans have also be used to measure molecular function and metabolic actions such as cerebral blood flow changes in brain regions associated with reward and arousal such as the ventral striatum, midbrain, amygdala, orbitofrontal cortex, and ventral medial prefrontal cortex are indicators of pleasurable experiences and can therefore also be used to correlate or validate Tingles or Flow States. Diffusion tensor imaging (DTI), fractional anistropy (FA) imaging, and Inter-beat intervals (IBIs) (an inverted measurement of heart rate), may also be used as additional methods of measuring or validating Tingles or Flow.

Gamma Waves

Our brainwaves change according to what we're doing, thinking and feeling. When slower brainwaves are dominant we can feel tired, slow, sluggish, or foggy. The higher frequencies are dominant when we feel energized, or hyper-alert. Gamma brainwaves are the fastest of brain waves and are related to processing of information from different areas in the brain simultaneously. The higher and lower frequencies link and cohere with each other through harmonics of all the frequencies. Researchers have discovered Gamma brainwaves are highly active when in states of universal love, appreciation, and the ‘higher virtues.’ Gamma is also above the frequency of neuronal firing, so how it is generated remains a mystery. It is speculated that gamma rhythms modulate perception and consciousness, and that a greater presence of gamma relates to expanded consciousness and spiritual emergence, which may be used to validate Flow States.

The embodiments herein expand on what is being measured by a Flow Sensor and distinguish the quality or value of different Flow states derived from different measurements. For example, some Flow states are more related to physical health and wellbeing or release of resistance while others may be experienced as peak physical performance and coordination, while others may be primarily mental in nature resulting in a stroke of genius insight, inspiration or clarity. Each has a different quality and these embodiments strive to distinguish between them by measuring different inputs on different parts of the body and/or using different methods in combination with each other to calibrate and validate Flow States for their type of Flow. Measurements from the heart field may differ in quality and value from those measured in brain waves. Furthermore, a Tingle state measured with the heart or solar plexus may have greater significance than a Tingle measured aurally with the ear or on the arms with a wrist band or bracelet. Brainwaves during a steady stream of gamma brainwaves that all of the sudden spikes may be able to distinguish the exact moment the person experiences a Eureka discovery moment in Flow and thereby validate the Eureka discovery itself and isolate these breakthroughs. Regardless of how Flow is measured, these embodiments may provide valuable feedback and couple the measurement with the issuance of a reward. The reward may be in the form of a currency, digital token, credit or donation. The reward may vary depending on the quality, duration, strength, timing, or type of Flow. Furthermore, these embodiments may associate or correlate the Flow with anything else, which may also participate in the rewards.

Flow Associator

The Flow sensor may also incorporate and communicate with a “Flow Associator,” which may be understood to be the electronics and software that determines or measures anything in time and space around the person in Flow by reading some form of Identification (ID) tags of anything on or near the person in Flow or by analyzing content the Flow user is exposed to or the Flow user's location, surroundings or activity. ID tags can be anything used for identification, e.g. RFID, barcode, QR code, audio/video/photo/pattern/facial recognition, etc. Alternatively, the sports equipment, vehicle or any product or person may have an ID tag built-in or attached.

The Flow Associator may pick up this product ID information, which may be built-into the Flow Sensor measuring device itself or provided separately in an accessory. In one example, Roger Federer is wearing a Flow measuring device which could be built-into his tennis racquet handle, headband, hat, visor, glasses, wristband, bracelet, watch, necklace, ring, shoes, clothing or placed anywhere on his body or a camera focused on his body and either that same device or an accessory built-into any of the foregoing includes the sensors for picking up ID tags to make the association of what or who corresponds to a person in Flow. As Roger Federer is playing tennis, any of the wearable Flow Sensors can measure and detect his Flow States and the Flow Associator can measure and detect any ID tags in and around him that may be correlated or associated with his achievement of a Flow State. The ID information and Flow status can be stored in any of the aforementioned local or wearable devices and communicated later in a store-and-forward manner or communicated in real time to a nearby communication device (e.g. cell phone, laptop, tablet) to a broader network, server, Blockchain, Internet, etc. for processing, further validation and the issuance of rewards, currency, digital or physical tokens, credits or donations, including the sharing of such token rewards with the correlated ID tag associations.

These new embodiments may be understood to be as broad as possible to include any and all physical objects, e.g. toys, books, all sports equipment (e.g. golf clubs, skis, snowboards, basketballs, tennis balls, baseballs, baseball and cricket bats, gloves, headbands, wristbands, footballs, soccer balls, surfboards, bicycles, shoes, clothing, computers, vehicles, motorcycles, skateboards, etc. to reward the Flow user and/or the provider or manufacturer of such tangible objects if such equipment can be associated with and/or contribute to the user achieving a Flow state. Such benefits could be further amplified by using Flow feedback to improve your timing, like knowing when you have achieved an optimal Flow State and then place a bet or invest in a certain stock or swing at the golf ball or when you are ready to get behind the wheel and drive your car or race the Formula 1. In general, calibrating and measuring yourself will lead to knowing where and under what conditions a persons Flow is stronger or more likely. The data would also be valuable to manufacturers in understanding which products and which conditions lead to better or lasting Flow states and are therefore more satisfying experiences for your customers.

These embodiments may be extended to confirm or validate the activity undertaken when users are using certain physical devices/equipment while in a coherent Flow state. For example, correlating the sweat absorbed by a head-band or wrist-band used to wipe sweat off users face coupled with a bracelet that reads your heart-beat increasing substantially while also detecting coherency in your heart field or brain waves (e.g. head-band) and/or or any number of ways to detect and correlate a person's activity with their Flow state. All of these inputs being used to further validate and correlate the athlete while actively playing a sport in a Flow State.

These embodiments include methods by which the users of Flow enriching products enter into sophisticated reward-sharing incentives with manufacturers. For example, when Roger Federer achieves a Flow state, he shares a portion of the tokens of himself with ON Shoes or vice-versa (e.g. he gets tokens of ON) or they split general purpose reward tokens issued or any complex model such as those used today by the financial markets in their elaborate profit-sharing models, e.g. min, max, carry-over, averages, bonus-matching, etc. He could even offer to share his tokens or ROGERs with fans in the stadium who achieve Flow with him. He could offer to share his ROGER tokens with his kids in Africa if they are watching and Flow with him. He could offer to share ROGERs with runners who get high running on clouds ON Running shoes.

Hardware manufacturers of all sorts of products may now have far greater incentives to deepen their relationship with customers in an on-going closer relationship of recurring value creation and sharing. Manufacturers may think differently about how to add value beyond the initial product sale, which may result in hardware being given away with greater focus on the back-end revenue streams. The back-end becomes the front-end. The vibration and integrity of products will begin to matter more in a new and more meaningful, rewarding and longer-term way. This may up-end the economics of hardware and give them new incentives to build better quality products. Now athletes like Megan Rapinoe can change the game and economics of sports. She can create tokens of herself when she Flows during a soccer/football match and scores a goal and then sell or share these tokens with fans watching in the stadium or at home. Flow Sensors level the playing field by leveling up and rewarding players who Flow. Flow Associators reward those who add value and contribute to their Flow or raise their vibration to match their true Flow.

In addition to physical goods, the Flow Associator embodiments contemplated herein include all content, e.g. videos, movies, audios, music, photos, articles, books, art-work, etc. to reward the creator of the content if such content can be associated with and/or contributes to the receiver achieving a coherent Flow State while experiencing or consuming their content. For example, you watch a video on YouTube. It might even be a pirated copy or was published without the creator's permission. Regardless, you watch it and you get Tingles—you achieve a Flow State as measured by your Flow Sensor. Furthermore, your Flow State is correlated with that particular video with your Flow Associator device that determines and validates the ID of the creator or author of the video. A token of your intrinsic value is created to represent and store the value of your Flow State. Furthermore, your token can be shared partially or wholly with the ID of the creator of the video. Additionally, or alternatively, a token of appreciation is created to represent the author/creator ID of the video content. Any combination of tokens can be created or transactions can be made to occur automatically between those who achieve Flow States and the people, products and services who are correlated to have contributed to the inspiration and influence of Flow States in others.

In another example, you could be having a conversation with someone or listening to a speaker on stage. Let's say all of the sudden while speaking or listening, you get Tingles. That is your indication of Flow and your Flow Sensor would measure and detect this and upon validating it, you would receive a token reward. Your Flow Associator would correlate with who you experienced this Flow State with the ID of the other person. Furthermore, the Flow Associator could determine who influenced who? Who was speaking? Who was listening? Who triggered who? The timing of events and their associations. The back-end Flow system could then issue additional tokens corresponding to the parties and benefits involved or share them accordingly.

Truth/Value Detectors

A person wearing a Flow Sensor could measure their flow rate or level of coherency and thereby their ability to calibrate to their “True” and best self. As explained in the previous embodiments, a person in Flow feels abundant, empowered, has tremendous clarity and insights into “win-win” solutions with a heart-centered mindset to the benefit of all parties involved, including those not immediately involved. The reason for this is because a person in the Flow is in resonance with their True selves and the True and best version of everyone else at the same time. That's why their ideas and creations resonate with everyone at their core at a soul level and are deeply valued by everyone. It would be extremely valuable to know when someone is in Flow as they are speaking, writing, creating or performing. They and others would know that whatever they just said, wrote or created had tremendous intrinsic true value. A person could easily assess themselves while they are thinking, imagining, reading, writing, creating, etc. Likewise, others are more likely to value your contributions more and feel better when they experience your expressions created in a Flow State.

As a listener or viewer, you may be able to detect the authentic value of the content if you are wearing a Flow Sensor and your body reacts; however, you might be out of Flow and not be able to resonate with the high frequency true value in that moment of absorbing the content. Furthermore, the true value of any content created depends on the authenticity of the creator, not the ability of the receiver to calibrate to it. When you are being true, it's about resonating with your own truth, not trying to please others or get their validation. Others might not be able to feel it or might even feel repelled by your frequency if they are feeling out of their true alignment. For example, let's say you are writing an Email message or a news article or a script for a story and you were wearing a Flow Sensor while expressing yourself. The content you create would calibrated to match your calibration.

If your Flow State is high while creating, the content could be authenticated and validated as being higher true value and rated higher for a win-win “Truth”. Your Emails could be “stamped” with a calibration matching your state of heart or mind while writing the Email. If you were in a Flow State while writing the Email, this would correlate to higher value and higher Truth. An Email server or inbox filter could filter Emails and put those on top of your inbox with higher Flow rates to prioritize Emails that correlate to the highest Truth and value in accordance with the author who wrote the Email. Similarly, search engines could prioritize and put articles on the front/top page that calibrate to a higher Truth and value by measuring the Flow Sensor of the author while s/he wrote the article and using a Flow Associator to correlate the author and his/her state when s/he created the content.

Search engines and content servers could more reliably detect fake news, or lower quality articles and videos that don't resonate with win-win truths or values. One of the greatest challenges for Google, Facebook and YouTube is to determine what content to block without censoring or encroaching on freedom of speech. Using the methods described herein to detect Truth, search engines could filter out false information based on poor Flow correlation between the author and what s/he produces. Search engines could also better match content vibration with persons doing searches by measuring the Flow State of persons doing the searches with those who create content. Search engines could detect the true calibration value of content. Search engines could better match up people with content that matches their vibration. Search engines could dynamically and intentionally challenge your beliefs by giving you results that rank slightly higher on a Truth scale (e.g. higher Flow State in the creator when the content was produced) as compared to your current Flow State and thereby offer upliftment or expansion of your beliefs.

Certain highly qualified persons who are particularly astute at holding steady high frequency Flow states while observing content created by others could provide a valuable service of measuring the authenticity of content in a manner similar to how Magnetic Resonant Imaging (MRI) works. Such persons could radiate their Flow state energy towards the content under observation and see/feel what they get back and compare the differences from True alignment.

Thought/Feeling Resonator

A resonator could be used to offer yourself better “feeling” thoughts when your own thoughts are transmitted from your own mind or received from others. For example, let's say you are wearing a “thought-translator” device or embedded in your mind or body. Such a device could discern certain thought patterns and further determine their value in terms of better feeling, uplifting, heart-opening, higher perspective, higher frequency, greater resonance with others, etc. Artificial intelligence could provide finer tuning, amplification, or better clarity to isolate and radiate the thoughts with higher value and better feelings. Similarly, such a device could tune out negative thought patterns and filter out those with lower frequency, quality and value. In either case, the feedback would help users resonate with their true selves.

Example Summary of the Embodiments

Regardless of how someone achieves a Flow State and regardless of how such a state is measured or validated, the embodiments described in U.S. provisional patent Application Number 2787078 may result in a reward of some kind. These embodiments may now encompass eleven (12) new broad solutions that have never been realized before:

(1) Methods for measuring and validating when a person is in a Flow State;
(2) Methods for associating a person in a Flow State with whatever or whoever may have contributed to or be correlated to that person in a Flow State;
(3) Methods for rewarding people in Flow with the products, content creators, services or companies who are associated with person(s) achieving Flow State(s);
(4) Methods for distinguishing the quality or type of Flow State achieved based on different measurements, characteristics, feelings or type of Flow Sensors or parts of the body involved;
(5) Methods for providing feedback on Flow State(s), including replays;
(6) Methods for validating or weighing votes, thoughts, or opinions of co-workers, Shareholders, Board Members or citizens to a higher degree when coupled with Flow State(s);
(7) Methods for inspiring and rewarding employees and consultants for their valuable contributions while in Flow States;
(8) Methods for ensuring wellbeing by rewarding wellness and positive events rather than insuring unwellness and rewarding illness, accidents and negative events;
(9) Methods for harnessing and storing the intrinsic value of Flow.
(10) Methods for measuring the truth, authenticity and value in any content as correlated with the degree to which the creator was in a Flow State when s/he created the content.
(11) Methods for filtering, prioritizing, blocking or promoting content based on the correlation of the creator of the content with their Flow State when the content was created.
(12) Methods for amplifying and focusing on thought patters with better feelings and filtering out and tuning out negative thought patterns.

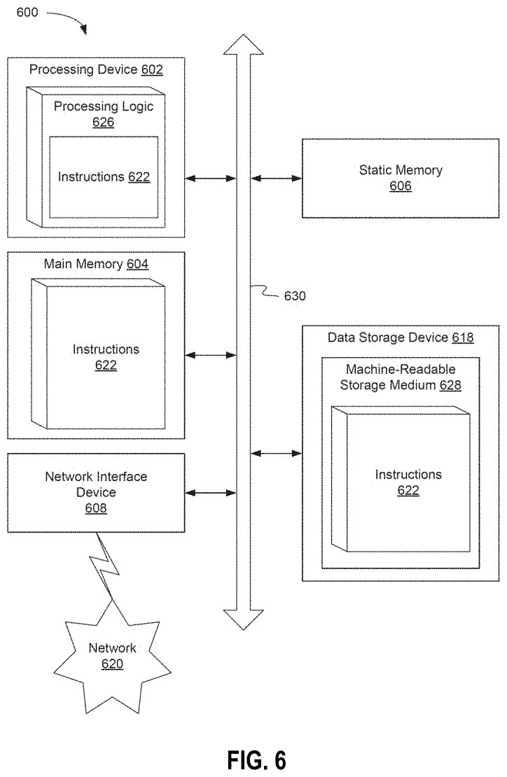

FIG. 6 is a block diagram of an example computing device 600 that may perform one or more of the operations described herein, in accordance with some embodiments. Computing device 600 may be connected to other computing devices in a LAN, an intranet, an extranet, and/or the Internet. The computing device may operate in the capacity of a server machine in client-server network environment or in the capacity of a client in a peer-to-peer network environment. The computing device may be provided by a personal computer (PC), a set-top box (STB), a server, a network router, switch or bridge, or any machine capable of executing a set of instructions (sequential or otherwise) that specify actions to be taken by that machine. Further, while only a single computing device is illustrated, the term “computing device” may also be taken to include any collection of computing devices that individually or jointly execute a set (or multiple sets) of instructions to perform the methods discussed herein.

The example computing device 600 may include a processing device (e.g., a general purpose processor, a PLD, etc.) 602, a main memory 604 (e.g., synchronous dynamic random access memory (DRAM), read-only memory (ROM)), a static memory 606 (e.g., flash memory and a data storage device 618), which may communicate with each other via a bus 630.

Processing device 602 may be provided by one or more general-purpose processing devices such as a microprocessor, central processing unit, or the like. In an illustrative example, processing device 602 may comprise a complex instruction set computing (CISC) microprocessor, reduced instruction set computing (RISC) microprocessor, very long instruction word (VLIW) microprocessor, or a processor implementing other instruction sets or processors implementing a combination of instruction sets. Processing device 602 may also comprise one or more special-purpose processing devices such as an application specific integrated circuit (ASIC), a field programmable gate array (FPGA), a digital signal processor (DSP), network processor, or the like. The processing device 602 may be configured to execute the operations described herein, in accordance with one or more aspects of the present disclosure, for performing the operations and steps discussed herein.

Computing device 600 may further include a network interface device 608 which may communicate with a network 620. The computing device 600 also may include a video display unit 610 (e.g., a liquid crystal display (LCD) or a cathode ray tube (CRT)), an alphanumeric input device 612 (e.g., a keyboard), a cursor control device 614 (e.g., a mouse) and an acoustic signal generation device 616 (e.g., a speaker). In one embodiment, video display unit 610, alphanumeric input device 612, and cursor control device 614 may be combined into a single component or device (e.g., an LCD touch screen).

Data storage device 618 may include a computer-readable storage medium 628 on which may be stored one or more sets of instructions, e.g., instructions for carrying out the operations described herein, in accordance with one or more aspects of the present disclosure. Instructions 622 implementing embodiments described herein may also reside, completely or at least partially, within main memory 604 and/or within processing device 602 (e.g., executed by processing logic 626) during execution thereof by computing device 600, main memory 604 and processing device 602 also constituting computer-readable media. The instructions 622 may further be transmitted or received over a network 620 via network interface device 608.

While computer-readable storage medium 628 is shown in an illustrative example to be a single medium, the term “computer-readable storage medium” should be taken to include a single medium or multiple media (e.g., a centralized or distributed database and/or associated caches and servers) that store the one or more sets of instructions. The term “computer-readable storage medium” may also be taken to include any medium that is capable of storing, encoding or carrying a set of instructions for execution by the machine and that cause the machine to perform the methods described herein. The term “computer-readable storage medium” may accordingly be taken to include, but not be limited to, solid-state memories, optical media and magnetic media.

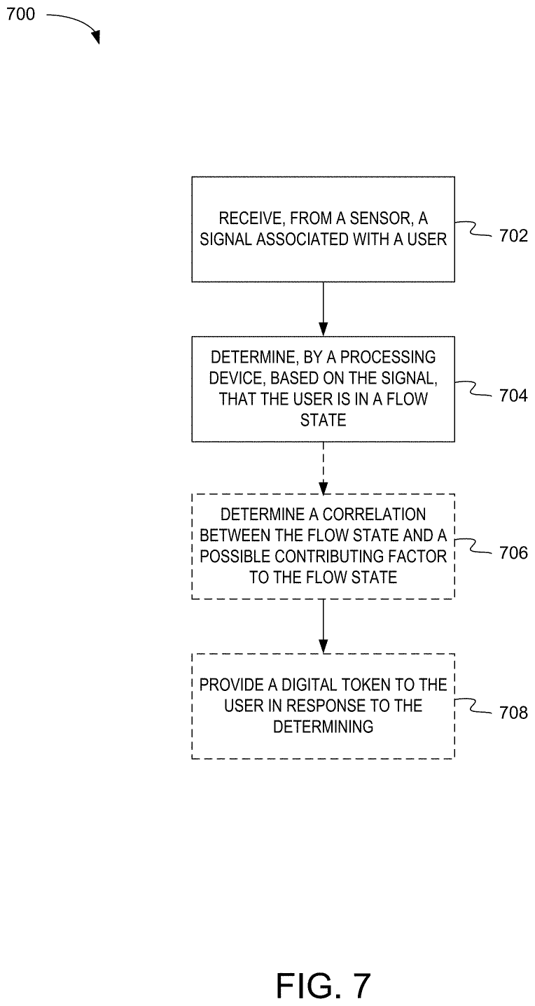

FIG. 7 is a flow diagram of method that may perform one or more of the operations described herein, in accordance with some embodiments. The method 700 may be performed by processing logic that comprises hardware (e.g., circuitry, dedicated logic, programmable logic, microcode, etc.), software (e.g., instructions run on a processing device to perform hardware simulation), or a combination thereof. In one embodiment, processing logic corresponding to one or more components described with respect to other figures may perform one or more of the following operations. For example, in one embodiment, processing logic of processing device 602 performs the following operations. In another embodiment, any other suitable processing device may perform the described operations.

Referring to FIG. 7, at block 702, processing logic may receive, from a sensor, a signal associated with a user. In one embodiment, as described herein, the sensor is located on a wearable of the user. In other embodiments, the sensor may be located in any physical object, including automotive components, furniture, compute devices, etc. In one embodiment, as described herein, the signal is an electrical signal, audio or video signal, magnetic signal, biological or chemical signal, brainwave signal, etc.

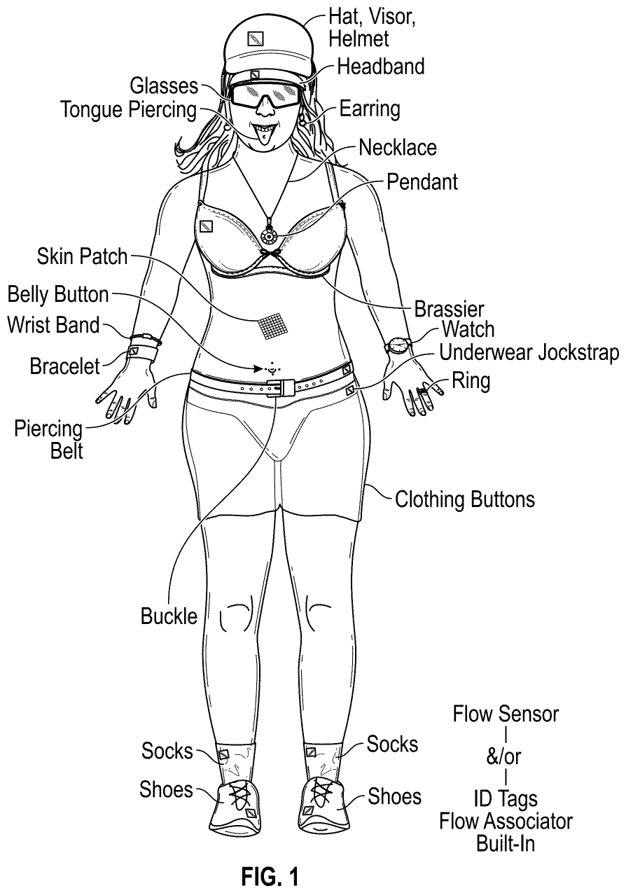

At block 704, processing logic may determine, by a processing device, based on the signal, that the user is in a flow state. At block 706, processing logic may determine a correlation between the flow state and a possible contributing factor to the flow state. In one embodiment the determination and correlation steps may be performed by any of the methods and systems described herein with respect to FIGS. 1-6 and the provided examples. In one embodiment, the correlation includes an indication of at least one of: a time, location, or experience associated with the flow state. In another embodiment, the correlation includes an indication of at least one of: a duration, intensity, or characterization of the flow state.

In another embodiment, processing logic may perform any other operations described herein. For example, processing logic may provide a digital token (e.g., on a blockchain) to the user in response to the determining.

The methods and illustrative examples described herein are not inherently related to any particular computer or other apparatus. Various general purpose systems may be used in accordance with the teachings described herein, or it may prove convenient to construct more specialized apparatus to perform the required method steps. The required structure for a variety of these systems will appear as set forth in the description above.

The above description is intended to be illustrative, and not restrictive. Although the present disclosure has been described with references to specific illustrative examples, it will be recognized that the present disclosure is not limited to the examples described. The scope of the disclosure should be determined with reference to the following claims, along with the full scope of equivalents to which the claims are entitled.

As used herein, the singular forms “a,” “an” and “the” are intended to include the plural forms as well, unless the context clearly indicates otherwise. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. Therefore, the terminology used herein is for the purpose of describing particular embodiments only and is not intended to be limiting.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Although the method operations were described in a specific order, it should be understood that other operations may be performed in between described operations, described operations may be adjusted so that they occur at slightly different times or the described operations may be distributed in a system which allows the occurrence of the processing operations at various intervals associated with the processing.

Various units, circuits, or other components may be described or claimed as “configured to” or “configurable to” perform a task or tasks. In such contexts, the phrase “configured to” or “configurable to” is used to connote structure by indicating that the units/circuits/components include structure (e.g., circuitry) that performs the task or tasks during operation. As such, the unit/circuit/component can be said to be configured to perform the task, or configurable to perform the task, even when the specified unit/circuit/component is not currently operational (e.g., is not on). The units/circuits/components used with the “configured to” or “configurable to” language include hardware—for example, circuits, memory storing program instructions executable to implement the operation, etc. Reciting that a unit/circuit/component is “configured to” perform one or more tasks, or is “configurable to” perform one or more tasks, is expressly intended not to invoke 35 U.S.C. 112, sixth paragraph, for that unit/circuit/component. Additionally, “configured to” or “configurable to” can include generic structure (e.g., generic circuitry) that is manipulated by software and/or firmware (e.g., an FPGA or a general-purpose processor executing software) to operate in manner that is capable of performing the task(s) at issue. “Configured to” may also include adapting a manufacturing process (e.g., a semiconductor fabrication facility) to fabricate devices (e.g., integrated circuits) that are adapted to implement or perform one or more tasks. “Configurable to” is expressly intended not to apply to blank media, an unprogrammed processor or unprogrammed generic computer, or an unprogrammed programmable logic device, programmable gate array, or other unprogrammed device, unless accompanied by programmed media that confers the ability to the unprogrammed device to be configured to perform the disclosed function(s).

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the embodiments and its practical applications, to thereby enable others skilled in the art to best utilize the embodiments and various modifications as may be suited to the particular use contemplated. Accordingly, the present embodiments are to be considered as illustrative and not restrictive, and the invention is not to be limited to the details given herein, but may be modified within the scope and equivalents of the appended claims.

The various components of embodiments described herein may comprise various construction materials. For example, each of the components may be constructed from injection mold plastic, 3-D printed plastic, wood, fiberglass, metal, cardboard, foam, rubber, carbon fiber, etc. Various coatings and/or coverings such as felt, velvet, rubberized paint, plastic, PVC, glass, foam, etc., may be applied to a base construction material to form the final components. Furthermore, any fastener type may be used in place of the fasteners described herein for convenience.

In the description herein, numerous specific details are set forth, such as examples of specific hardware structures, specific architectural and micro architectural details, specific components, specific measurements/heights, etc. in order to provide a thorough understanding of the present disclosure. It will be apparent, however, that these specific details need not be employed to practice the present disclosure. In other instances, well known components or methods, such as specific and alternative construction materials, dimensions, shapes, sizes, functions and other specific details of the various versions of the apparatus described herein have not been described in detail in order to avoid unnecessarily obscuring the present disclosure.

Use of the phrases ‘to,’ ‘capable of/to,’ and or ‘operable to,’ in one implementation, refers to some apparatus, system, component, member, and/or element designed in such a way to enable use of the apparatus, system, component, member, and/or element in a specified manner. Note as above that use of ‘to,’ ‘capable to,’ or ‘operable to,’ in one implementation, refers to the latent state of an apparatus where the apparatus is not operating but is designed in such a manner to enable use of an apparatus in a specified manner.

Reference throughout this specification to “one embodiment” or “an embodiment” means that a particular feature, structure, or characteristic described in connection with the embodiment is included in at least one embodiment of the present disclosure. Thus, the appearances of the phrases “in one embodiment” or “in an embodiment” on “in some embodiment” in various places throughout this specification are not necessarily all referring to the same embodiment. Furthermore, the particular features, structures, or characteristics may be combined in any suitable manner in one or more embodiment.

In the foregoing specification, a detailed description has been given with reference to specific exemplary implementations. It will, however, be evident that various modifications and changes may be made thereto without departing from the broader spirit and scope of the disclosure as set forth in the appended claims. The specification and drawings are, accordingly, to be regarded in an illustrative sense rather than a restrictive sense. Furthermore, the foregoing use of implementation and other exemplarily language does not necessarily refer to the same implementation or the same example, but may refer to different and distinct implementations, as well as potentially the same implementation.

The words “example” or “exemplary” are used herein to mean serving as an example, instance or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an embodiment” or “one embodiment” or “an embodiment” or “one embodiment” throughout is not intended to mean the same embodiment or embodiment unless described as such. Also, the terms “first,” “second,” “third,” “fourth,” etc. as used herein are meant as labels to distinguish among different elements and may not necessarily have an ordinal meaning according to their numerical designation.

### Further Non-Limiting Examples

It is contemplated that a person having ordinary skill in the art would be able to understand and construct each of the embodiments and examples described herein. Non-limiting examples, in addition to those described above, are provided below:

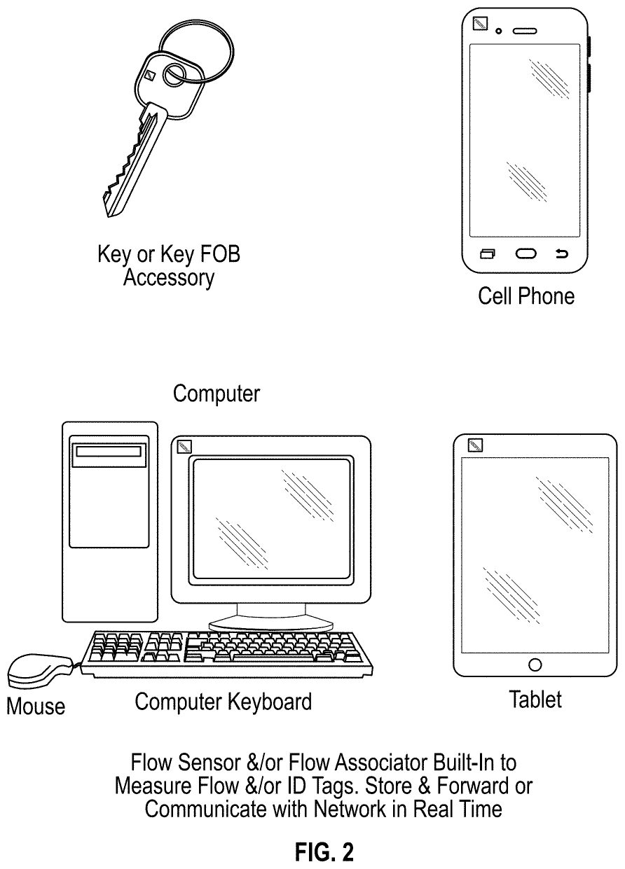

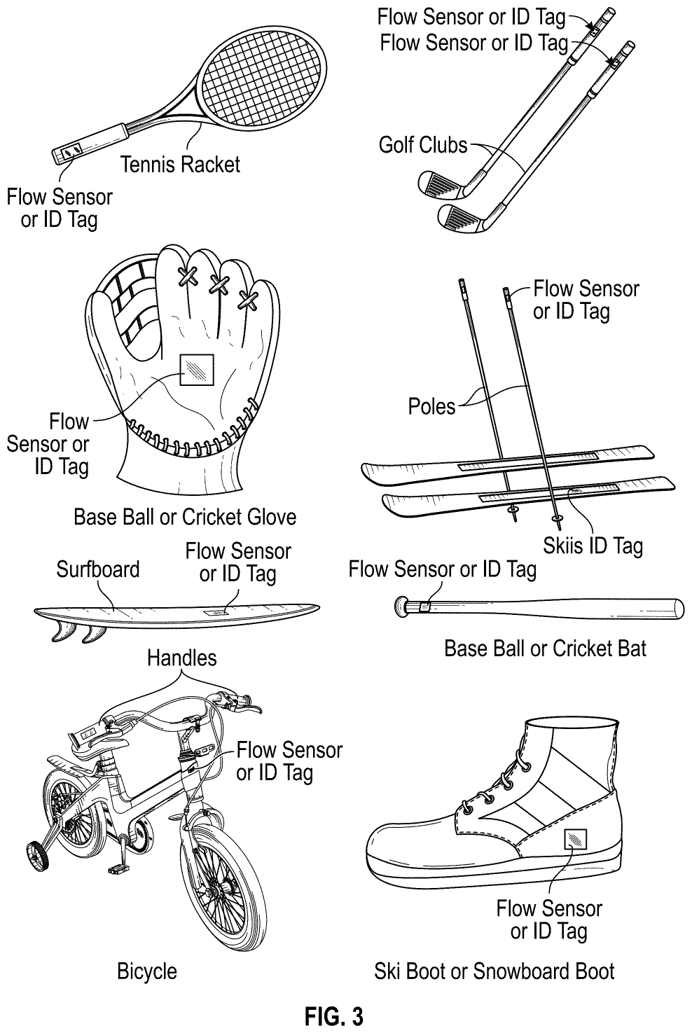

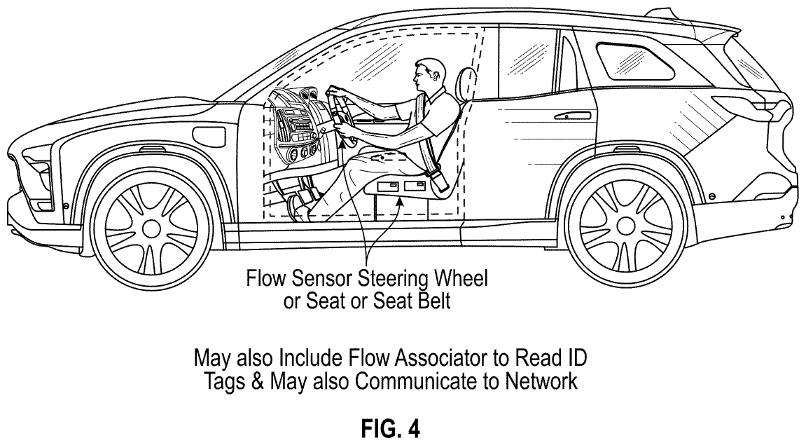

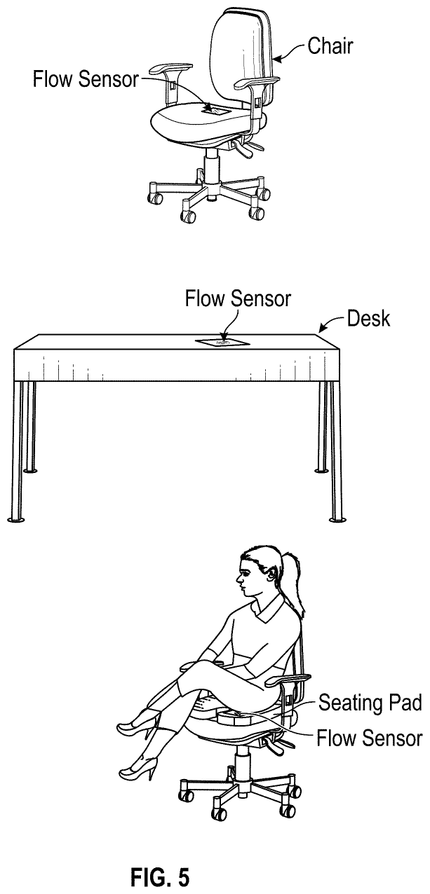

An example of measuring and validating Flow State in a person by any means of measurement(s), or combinations thereof, including but not limited to the following:
i. Heart-beat rate and/or heart rate variability (e.g. Heartmath);
ii. Magnetic fields of any cells or organs in the human body (e.g. the heart field, solar plexus, brain, hypothalamus, amygdala, etc.);
iii. Brain wave activity or frequency (e.g. gamma waves, determining thought content or patterns, etc.);
iv. Solar plexus gut activity/signals;
v. Cellular, hormonal, or neurotransmitter activity/signals;
vi. Pupil dilation;
vii. Electrodermal activity (EDA);
viii. Skin Conductance Response (SCR) and in particular low or high resistance, high or low capacitance and surges or pulses of energy resulting in sudden or dramatic changes in resistance or capacitance of the skin;
ix. Electrical or magnetic potential differences or dramatic changes in electrical or magnetic conductivity or potential differences inside or on the body;
x. Changes in resistance on the skin or any body part inside or out;
xi. Cellular, molecular or organ frequency, resonance and/or radiance of magnetic, electrical or light frequencies;
xii. Cellular, molecular or organ bioluminescence;
xiii. Deformation of skin or hairs using capacitance, e.g. spiral coplanar capacitors;
xiv. Stick-on transparent conductive polymer to quantify intensity and/or duration by recording a drop in capacitance and its ability to store an electrical charge caused by a deformation or buckling of the skin's surface;
xv. Spatiotemporal decomposition of electromyographical signals to isolate piloerection signal from surrounding noise;
xvi. Interpretation of physical senses, blood quality or blood flow using Positron emission tomography (PET) scans or Magnetic Resonance Imaging (MRI) to measure molecular function and metabolic actions such as cerebral blood flow changes in the body, heart or brain regions associated with reward and arousal such as the ventral striatum, midbrain, amygdala, hypothalamus, orbitofrontal cortex, or ventral medial prefrontal cortex;
xvii. Diffusion tensor imaging (DTI) or Fractional anistropy (FA) imaging;
xviii. Inter-beat intervals (IBIs), an inverted measurement of heart rate;
xix. Analysis of frequency, harmonics and coherency in voice tones;
xx. Kirlian or aura photography;
xxi. Voltage, electrical or magnetic differences in the body between head and grounded feet;
xxii. Spontaneous body movements or release of energy or resistance;
xxiii. Kinesiology or muscle testing;
xxiv. Body and brain chemistry to determine the presence of certain hormones, neurotransmitters, endorphins, oxytocin, negative ions and to rule out chemically induced reactions;
xxv. Electrical measurements to rule out electrically induced reactions;
xxvi. Body temperature in part to rule out environmental inducements, fear or fever;
xxvii. Blood composition, blood flow, density or analysis.
2. An example of example 1 where the Flow State is measured by a Flow Sensor worn by the user and contained in any one or more of the following:
   i. Necklace/pendant (see FIG. 1)
   ii. Headband (see FIG. 1)
   iii. Hat, helmet or visor (see FIG. 1)
   iv. Glasses, sunglasses, contact lenses, eye implants (see FIG. 1)
   v. Earrings or ear piece (see FIG. 1)
   vi. Rings (see FIG. 1)
   vii. Piercings (see FIG. 1)
   viii. Wristband (see FIG. 1)
   ix. Bracelet (see FIG. 1)
   x. Watch (see FIG. 1)
   xi. Belt (see FIG. 1)
   xii. Belt-buckle (see FIG. 1)
   xiii. Buttons (see FIG. 1)
   xiv. Clothing, including tags or attachments to clothing (see FIG. 1)
   xv. Brassier, underwear or jockstrap (see FIG. 1)
   xvi. Patch worn on the skin or implanted inside the body (see FIG. 1)
   xvii. Shoes or socks (see FIG. 1)
   xviii. Key fob/accessory held near the body (see FIG. 2)
   xix. Cell phone (see FIG. 2)
   xx. Computer or tablet (see FIG. 2)
   xxi. Computer mouse, mousepad or keyboard (see FIG. 2)
   xxii. Built directly into sports equipment, handle, glove (see FIG. 3)
   xxiii. Steering wheel, seat or seat belt of a vehicle (see FIG. 4)
   xxiv. Desk or chair or seating pad, bed or bed sheets (see FIG. 5)
3. An example of example 1 and/or example 2 wherein the Flow State of a person is associated with time, space, place, things, content, activities or other people.
4. An example of example 1 and/or example 2 independently or any combination of methods or Claims herein where the correlation is a person's physical location on earth or anywhere in space using GPS or any other location sensing mechanism. For example, the person is in a forest, in the mountains, in a stadium, at a hotel, at home, moving in a car, tram, bus, train, bike etc., when they reach a Flow State.
5. An example of example 1 and/or example 2 independently or any combination of methods or Claims herein where the correlation is the time of day and/or the date in the location where the person in Flow takes place.
6. An example of example 1 and/or example 2 independently or any combination of methods or Claims herein where the correlation is made with an audio input, listening or recording device to identify someone or something audibly is playing in the vicinity or remotely listening to during or prior to a person reaching a Flow State and then identifying the voice(s) and the people, place, content or activity. For example, a person in a Flow State is listening to a particular song or teacher online or in person during or just prior to reaching a Flow State or a person is in a meeting with their boss or on the phone with their lover when s/he reaches a Flow State.
7. An example of example 1 and/or example 2 independently or any combination of methods or Claims herein where the correlation is made with video input or video streaming or recording device to identify something visually dynamic or changing patterns are playing in the vicinity or watching remotely during or prior to a person reaching a Flow State and then identifying the person, place or content. For example, a person in a Flow State is watching Roger Federer play tennis on TV, online or in person. Or a person is watching a movie or TV show during or just prior to reaching a Flow State and identifying the movie as the Color Purple and the actress speaking was Oprah Winfrey or the TV show was Greys Anatomy and the actress speaking was Ellen Pompeo.
8. An example of example 1 and/or example 2 independently or any combination of methods or Claims herein where the correlation is made with a photographic, camera or microscopic imaging device to identify something visually that is static or changing slowly in the vicinity or being looked at remotely during or just prior to a person reaching a Flow State. For example, a person takes photo(s) or looks at photo(s) and achieves a Flow State or responds to a text message or posts a photo on social media when s/he reaches Flow State and correlating the photo and/or person, place or thing in the photo as being associated with the Flow State.
9. An example of example 1 and/or example 2 independently or any combination of methods or Claims herein where the correlation is the duration of time in which the Flow State lasts.
10. An example of example 1 and/or example 2 independently or any combination of methods or Claims herein where the correlation is the intensity or magnitude of Flow experienced in coherency, resonance or any method of measuring significance, strength or amplitude.
11. An example of example 1 and/or example 2 independently or any combination of methods or Claims herein where the correlation is the type of Flow achieved based on what is being measured to ascertain the kind of Flow being experienced, e.g. relief, reduction in resistance, physical or general wellbeing, positive emotions (joy or love), heightened mental clarity, a moment of genius, great discovery or hyper-intelligence, or spontaneous spiritual awakening. For example, a spike in gamma waves being used to distinguish mental Flow clarity from heart-field magnetic resonance being used to determine love Flow or SCR being used to validate reduced resistance or Tingles in one's ears, solar plexus or forearms. Using this information to derive feedback and any variation in rewards resulting from such differences. For example, a low-level Flow triggered by moving up the emotional scale from depression to anger is different than moving from overwhelment to satisfaction or from joy to love or bliss.
12. An example of example 1 and/or example 2 independently or any combination of methods or Claims herein where the activity of a person in Flow state is derived by what is on their calendar, interpretation of any text or email messages, or any combination of video or audio inputs, or photos taken or location identification inputs (e.g. GPS on mobile phone) and any intelligence or data used to correlate any of this information to ascertain the activity of a person while achieving a Flow State, including validation or inputs by others.
13. An example of example 1 and/or example 2 independently or any combination of methods or Claims herein where the activity of a person in Flow state is derived and correlated with their Flow State. For example, were they meditating, sleeping, reading, listening to music, cooking, eating, drinking, under the influence of drugs or alcohol, driving, having sex (by themselves or with others), being physically active at the gym or outdoors riding a bike or playing sports or inside concentrating at their computer.
14. An example of example 1 and/or example 2 independently or any combination of methods or Claims herein where the identification of any person(s) who is physically near a person in Flow state is derived and who may be influencing them. For example, PINK is identified as the artist singing at a concert while you are in attendance and reach a Flow State while listening to her at the same concert. This could be determined by taking a selfie of yourself on a mobile phone with a QR code or PINK herself projected on the screen above the concert stage. The software would validate your location and confirm your presence with PINK at a particular event and your Flow state. Or, you are having an inspiring and uplifting conversation with someone who contributes to your reaching a Flow State or vice-versa. An algorithm to determine who is influencing who more using timing, duration, intensity, proximity or activity.
15. An example of example 1 and/or example 2 independently or any combination of methods or Claims herein where a person in a Flow State is further correlated with any physical object near them or on them or in their possession by means of a Flow Associator identifying the “ID” tags (e.g. RFID, barcode, QR code, nano-chips) on goods near or on the person in Flow or based on chemistry measurements, mass spectrometry, blood content, electromagnetic measurements, or photos, videos or audios being interpreted using pattern recognition software. For example, Roger Federer is holding a Wilson tennis racquet and wearing Nike tennis shoes and UNIQLO clothing and wearing a Rolex watch while playing tennis in a Flow State, all of which could be picked up by an RFID reader or any ID reader in a Flow Associator, either built into the same product as the Flow Sensor (any described in claims 1 and 2) or separately in a Flow Associator also in any form of claim 1 or 2 such as a watch, necklace, headband, wristband, or anywhere on or near his body. Such information could then be locally stored and/or communicated over any wired or wireless methods (e.g. cell phone, laptop, tablet, etc.) to a software server to further validate and/or provide feedback and/or rewards to the parties involved.
16. An example of example 1 and/or example 2 independently or any combination of methods or Claims herein where the physical, emotional or mental state of the person or activity is further determined by any of the methods in claim 1 and/or other claims herein including video, photo or pattern recognition, data inputs, or validation by sufficient witnesses.
17. An example of example 1 and/or example 2 independently or any combination of methods or Claims herein where a person in a Flow State is further correlated with the original author or creator of any content the Flow user may be receiving or be exposed to. For example, a person in a Flow State is listening to Ester Hicks on YouTube and Ester Hicks is identified and recognized as the original author of the content rather than the party who posted an unauthorized version of Ester Hicks on YouTube.
18. An example of example 1 and/or example 2 independently and any combination of methods or Claims herein whereby another party or a company is rewarded for their association or correlation to a person reaching a Flow State either by virtue of any the methods described herein or by virtue of who or what the person in Flow State chooses to share rewards with. For example, Roger could say today I am playing for my kids in Africa who are in Flow! Or PINK could say today I am Flowing to my fans in attendance at my concert tonight, especially anyone who Flows with her. Ellen Degeneres could say, anyone who Flows with me today during my show will receive a portion of my rewards or together we channel our collective rewards to the Ellen Fund to enhance the lives of endangered animals. Leonardo DiCaprio could say when you Flow with me, The Leonardo DiCaprio Foundation will be rewarded. Such examples further amplify the power of influence by the person Flowing sharing or issuing their rewards or tokens partially or fully with those who Flow with them at virtually the same time, or during the event or while watching a replay, and even from a distance.
19. An example of example 1 and/or example 2 independently and any combination of methods or Claims herein whereby two or more people are correlated together such as opponents in a match or the fans watching a match or a team of players or people and they are correlated as being in Flow together or influencing the other to achieve or sustain a Flow State.
20. An example of example 1 and/or example 2 independently and any combination of methods or Claims herein whereby a reward is created and/or issued in the form of a digital token, credit or donation or even a fiat currency or digital currency (e.g. crypto) for achieving Flow and/or being associated with someone in Flow. The reward could be a digital token of value on the Blockchain or the Internet, regardless of whether it is centralized or not, public or private. For example, Megan Rapino or Ada Hegerberg could say to their fans, if you Flow when I score a goal (or anytime while watching me), you get a portion of my rewards and/or you create and receive your own general purpose reward tokens or personal tokens. Megan could say “today I am Flowing with you. Are you?”
21. An example of example 20 whereby the digital token of value is a general purpose token of shared or common value (e.g. Token of Me, Mother Tree Foundation).
22. An example of example 21 whereby the digital token of value is specific to that person or company (e.g. ROGER token or WILSON token), regardless of whether you get a token of yourself or the other entity or party involved. For example, Roger Federer Flows with a Wilson tennis racquet and he creates a ROGER token and shares this partially or fully with Wilson (the Company), or he creates both a ROGER token and a WILSON token and he either receives both or shares them partially or fully with Wilson (the Company). Alternatively, he creates a general purpose token (e.g. Token of Me) and/or only a ROGER token and shares them partially or fully with Wilson (the Company).
23. An example of example 22 whereby the digital tokens are blended (e.g. ROGER/RAFA or the entire soccer/fussball team (e.g. the American Women World Cup Winners), especially when such players Flow simultaneously or during the same match. For example, all the players on a team who manage to achieve Flow during a match could merge their reward tokens into a blended commemorative digital token which can be further made into a physical token.
24. An example of examples 19-22 whereby the digital token of value is tied to the intrinsic or monetary value of that person or company or any interpretation of their outer value, inner value or ability to Flow.
25. An example of examples 19-23 whereby the digital token of value is exchanged for other digital tokens or sold in exchange for any currency (regardless of cryptographic or fiat currency) or anything tangible (e.g. stock, real estate, gems, metals, etc.) or traded as a credit or granted as a donation.
26. An example of examples 20-24 whereby the intangible digital token is made tangible in any physical form.
27. An example of example 26 whereby the intangible digital token is virtually dissolved, burned or melted and thereby no longer exists virtually once it is made tangible in a physical form.
28. An example of examples 20-27 whereby the person in Flow State gets to choose (or not) the revenue-sharing mix between themselves and all things correlated or associated with their reaching or sustaining a Flow State. For example, smart contracts are written up between advanced athletes in partnership with their sponsors and the tokens are issued automatically as contractually agreed.
29. An example of example 20-28 whereby a digital token transforms into another type of token. For example, someone named Alexandra Flows and by doing she initially creates and receives general purpose tokens (e.g. Token of Me) and after a certain event takes place or a certain number of TOM tokens are created, she has the option to transform some or all these into personal tokens of ALEXANDRA, the embodiment of her own inner personal value.
30. An example of example 48 whereby the personalized tokens of ALEXANDRA are backed by something else or blended like an alloy with another token of value. For example, ALEXANDRA tokens could be empowered with the value of another digital token of value, e.g. The Mother Tree Foundation, or any other digitized value like a stable coin, precious metal coins, shares, rights or values in a company or entity.
31. An example of example 1 and/or example 2 independently and any combination of methods or Claims herein whereby the contents, maker or brand of what someone is eating or drinking in Flow State becomes known by virtue of any means and a correlation is made with the person in Flow and the digestible food or drink they consumed while Flowing. The contents could be determined by blood analysis or stomach contents or vibrationally measuring (e.g. mass spectrometry) or nano ID chips in the food or liquid or the wrapper. For example, Lindt or Nestle could create a new chocolate bar or candy with microscopic biodegradable ID chips in the wrapper and/or the food itself to be identified in association with a person in a Flow state while consuming.
32. An example of example 31 whereby a token of value is issued in accordance with any of the claims 20-30.
33. An example of example 1 and/or example 2 independently or any combination of methods or Claims herein where fraud is detected to invalidate the Flow and/or issuance of a token, whether digital or physical.
34. An example of example 1 and/or example 2 independently or any combination of methods or Claims herein where false-positive(s) are detected in Flow and used to invalidate or prevent the issuance of a token, whether digital or physical. For example, a person has a fever and this triggers chills which are not based on emotions. Or, a person holds an ice cube to trigger chills which is not reflective of emotions. Or a person triggers goosebumps by digesting chemicals or drugs or electrically on their skin.
35. An example of example 1 and/or example 2 independently and any combination of methods or Claims herein whereby the information is used to derive any variation in rewards resulting from such differences.
36. An example of example 1 and/or example 2 independently and any combination of methods or Claims herein whereby the person in Flow shares their Flow data with anyone or any company whose products or services are correlated with their Flow and does so either transparently or anonymously, regardless of whether tokens are issued or shared with such parties.
37. An example of example 1 and/or example 2 independently and any combination of methods or Claims herein whereby an insurance company lowers the premiums for a person based on their Flow data. For example, health insurance premiums are lowered for people with higher Flow results or automobile insurance is lower for drivers with high Flow levels while driving.
38. An example of example 1 and/or example 2 independently and any combination of methods or Claims herein whereby an insurance or ensurance company actually pays or rewards someone based on their Flow data and in particular positive Flow results. By ensurance, I mean ensuring wellbeing rather than insuring against unwell-being or accidents. For example, instead of paying for insurance to pay out when unwanted happens, the ensurance company pays out when wanted Flow or desired events happens.
39. An example of example 1 and/or example 2 independently and any combination of methods or Claims herein whereby an employer offers bonuses or salaries based on an employee or consultant's Flow results. For example, a company could offer its employees a Flow token bonus matching program. For every Flow token created (while working or not) and made publicly trade-able, they will match it by buying it on the open market and paying their staff a salary or bonus for achieving Flow.
40. An example of example 1 and/or example 2 independently and any combination of methods or Claims herein whereby an employer offers bonuses or salaries based on an employee or consultant's Flow results in a team or in partnership with another to actively encourage positively influencing others while you are in Flow.
41. An example of example 1 and/or example 2 independently and any combination of methods or Claims herein whereby a person or a company offers payment, revenue-sharing or token-sharing based on Flow results of the other party or correlated with the parties involved.
42. An example of example 1 and/or example 2 independently and any combination of methods or Claims herein whereby the vote of a board member or shareholder in an entity or is only valid or weighted higher if/when a voter is in Flow when they cast their vote.
43. An example of example 1 and/or example 2 independently and any combination of methods or Claims herein whereby the vote of citizens in a country are only valid or are weighted higher if/when they are in Flow when they cast their vote.
44. An example of example 1 and/or example 2 independently and any combination of methods or Claims herein whereby any Flow Sensor and/or Flow Associator provides feedback by glowing or giving off light, sound, energy, vibration, sensation or change in temperature, color, sharpness or sound when Flow is approached, reached, validated or rewarded. For example, a necklace Flow Sensor may glow red when out of Flow and green or white when Flowing. Alternatively, an earring or ear piece may give off a distinct sound or vibration when in/out of Flow. Alternatively, a pair of glasses may respond optically with feedback on Flow States by changing its focal point or filter when moving in the direction of Flow or get fuzzy focus or dulling the colors when moving away from Flow. Alternatively, a patch worn on the skin or clothing or your shoes or socks may give off heat or a pleasant tingling sensation when Flowing and cold or unpleasant sensation when not Flowing. Such a device could be implanted inside your body, brain or heart like a pace-maker but instead would be a resonant-maker of truth.
45. An example of example 44 whereby the feedback indicators are the result of getting warmer as you move in the direction of Flow or someone else in Flow or of similar frequency moves closer to you to indicate a match similar to a metal-detector, only it's a resonate-detector of matching calibrations.
46. An example of example 44 whereby the feedback indicators are the result of moving away from Flow, losing Flow or lowering Flow or frequency mismatch.
47. An example of example 46 whereby a vehicle or an airplane will not start if you are particularly out of Flow and in the driver's seat or put your hand on the steering wheel or controls.
48. An example of example 46 whereby a vehicle provides feedback if you are driving and losing Flow or out of Flow. For example, your hazard lights might start blinking or the speed limit may be lowered.
49. An example of example 46 whereby your computer or mouse or keyboard or cell phone will not work or responds differently or provides less functionality if you are out of Flow.
50. An example of example 1 and/or example 2 independently and in combination of claims 44-46 whereby the feedback triggers an audio and/or photographic or video recording device on or near the user in Flow or out of Flow. For example, a person in Flow triggers a photo being taken or video camera embedded in their glasses or audio recording in their cell phone and such information is stored to be reviewed later when the user wants to know when they were in Flow or when they lost Flow and what was happening at that moment in time. The video, audio or photos may be correlated with the Flow State for positive and negative feedback and to relive the Flow moments.
51. An example of example 50 whereby win-win solutions or answers are delivered to a person in Flow through images, video or audio in any manner, e.g. future humans will be able to communicate telepathically. This means when someone is in Flow, they can more easily receive information from a computer or another person mentally even from a distance. This Claim intends to translate such information into images, videos or audio messages for tangible translations.
52. An example of examples 1 and/or 2 whereby a transducer is used to harvest and transform the high frequency or resonate energy flowing or focused through a person in Flow into electrical and/or magnetic energy or any other vibration.
53. An example of examples 1 and/or 2 whereby the action, interaction or output of the Flow Sensor is able to power the feedback mechanism or recharge or power the Flow Sensor or the Flow Associator or a cell phone or a pace-maker or anything else.
54. An example of examples 51 and 52 whereby energy produced through a Flow Sensor or

Flow Associator is stored in either one or transferred to another storage device.
55. Any combination of methods or Claims herein whereby a person votes electronically and his/her Flow state is recorded and thereby transmitted and used to weigh the vote, confirm it or negate it. In particular, the Flow state of the voter is used to authentic the vote itself as being true to the voter and thereby valid. In this way, the votes rendered by shareholders in companies could be weighed higher the truer the person was when they cast their vote in a Flow state. Votes could be weighed less or considered invalid if the person was below a certain Flow rate when they voted. In the inventor's company, shareholder's votes only count when the shareholder is in the Flow when they cast their vote.
56. Any combination of methods or Claims herein whereby a user is wearing a Flow Sensor while creating something and that something is then given a value rating that indicates the level of authenticity of the creation as determined by the Flow State of the creator while creating.
57. An example of example 56 whereby a search engine or filtering software ranks content created with higher Flow rates with higher visibility or higher ranking. Similarly, such a filter could be used to filter out or block content based on low Flow rates by the sender or creator.
58. An example of examples 56 and 57 whereby a software server, Email inbox, or text notification prioritizes messages from senders with higher Flow rates as being more authentic or more valuable or more important. Such a filter could be used to determine relevancy or win-win value or authenticity of the sender.
59. An example of examples 56-58 whereby a cell phone only allows calls or texts to come through from callers who are in a Flow state or filters out calls or text messages while the receiver is being more focused or calibrating to their true self.
60. An example of examples 56-59 whereby content is blocked or deprioritized or flagged by virtue of a particularly low Flow rate of the sender or creator of content.
61. Any combination of methods or Claims herein whereby your thought patterns are determined to be negative or positive and then the positive ones are amplified and the negative ones defused or dampened.
62. An example of example 61 whereby your feelings are determined to be good or bad based on your Flow Sensor and the good ones are amplified by positive feedback in the Flow Sensor and the bad ones are defused or dampened by negative feedback in the Flow Sensor or no feedback at all.

What is claimed is:

1. A method, comprising:
receiving, from a sensor, a signal associated with a user; and
determining, by a processing device, based on the signal, that the user is in a flow state.

2. The method of claim 1, wherein the sensor is located on a wearable of the user.

3. The method of claim 1, wherein the signal is an electrical signal.

4. The method of claim 1, wherein the signal is an audio signal.

5. The method of claim 1, wherein the signal is at least one of a magnetic signal or biological signal.

6. The method of claim 1, further comprising determining a correlation between the flow state and a possible contributing factor to the flow state.

7. The method of claim 1, further comprising determining a correlation between the flow state and a possible contributing factor to the flow state.

8. The method of claim 7, wherein the correlation comprises an indication of at least one of: a time, location, or experience associated with the flow state.

9. The method of claim 7, wherein the correlation comprises an indication of at least one of: a duration, intensity, or characterization of the flow state.

10. The method of claim 1, further comprising providing a digital token to the user in response to the determining.

11. A system, comprising:
a memory to store a signal associated with a user; and
a processing device operatively coupled to the memory, the processing device to:
receive, from the sensor, a signal associated with a user; and
determine based on the signal, that the user is in a flow state.

12. The system of claim 11, wherein the sensor is located on a wearable of the user.

13. The system of claim 11, wherein the signal is at least one of: an electrical signal, audio signal, magnetic signal, or biological signal.

14. The system of claim 11, the processing device further to determine a correlation between the flow state and a possible contributing factor to the flow state.

15. The system of claim 11, the processing device further to determine a correlation between the flow state and a possible contributing factor to the flow state.

16. The system of claim 15, wherein the correlation comprises an indication of at least one of: a time, location, or experience associated with the flow state.

17. The system of claim 15, wherein the correlation comprises an indication of at least one of: a duration, intensity, or characterization of the flow state.

18. The system of claim 1, the processing device further to provide a digital token to the user in response to the determining.

19. A non-transitory computer-readable storage medium comprising instructions, which when executing by a processing device, cause the processing device to:
receive, from a sensor, a signal associated with a user; and
determine based on the signal, that the user is in a flow state.

20. The non-transitory computer-readable storage medium of claim 19, the processing device further to determine a correlation between the flow state and a possible contributing factor to the flow state, wherein the correlation comprises an indication of at least one of: a time, location, experience, duration, intensity, or characterization associated with the flow state.
